(19) United States
(12) Patent Application Publication
(10) Pub. No.: US 2016/0263168 A1
(43) Pub. Date: Sep. 15, 2016

(54) Babies' Magic Tea

(71) Applicant: Samah Bensalem, Aventura, FL (US)

(72) Inventor: Samah Bensalem, Aventura, FL (US)

(21) Appl. No.: 15/055,570

(22) Filed: Feb. 27, 2016

Related U.S. Application Data
(60) Provisional application No. 62/110,864, filed on Feb. 2, 2015.

(51) Int. Cl. A61K 36/235; A23C 9/152; A23F 3/34; A61K 36/23

(52) U.S. Cl. CPC A61K 36/235; A61K 36/23; A23C 9/152; A23F 3/34

(57) ABSTRACT

A method of preparing tea comprising steps (a) selecting raw materials, wherein the raw materials include organic cumin seeds and organic funnel seeds; (b) washing the raw materials thoroughly; (c) sterilization of the raw materials via air-drying; (d) mixing a predetermined amount of organic funnel seeds and a predetermined amount of organic cumin seeds into a tea bags, wherein the tea bag is 1.5 g; (e) boiling 8 ounces of water, wherein the tea bag is added to the boiling water for 2 to 3 minutes; and (f) serving the tea to a baby at room temperature wherein the tea is intended for babies to reduce colic, gas, constipation, and acid reflux.

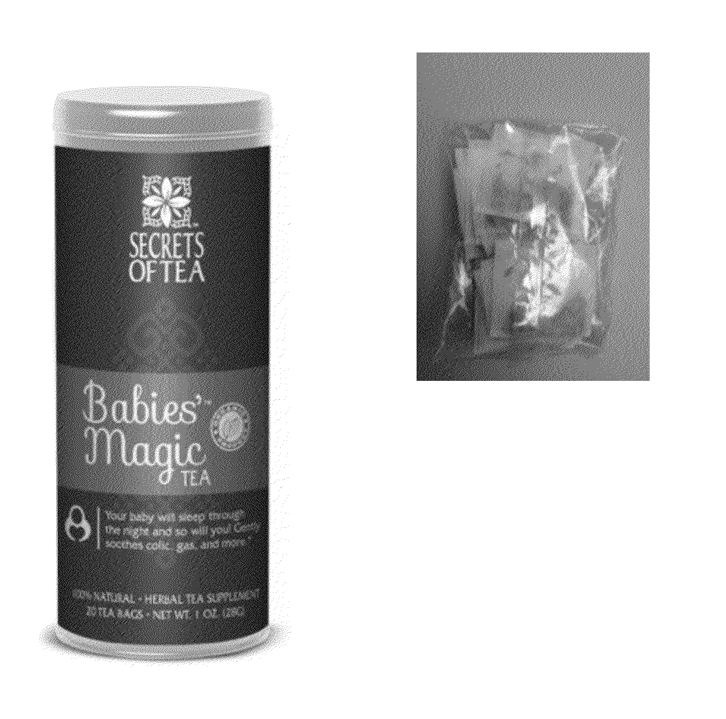

### CROSS-REFERENCE TO RELATED APPLICATIONS

N/A

### BACKGROUND OF THE INVENTION

The present invention relates to a proprietary blend of herbal tea for babies.

### BRIEF SUMMARY OF THE INVENTION

Babies Magic Tea is an herbal tea that contains two ingredients, organic cumin seeds and organic funnel seeds, the combination between these two ingredients is very effective to treat baby colic, gas, constipation, acid reflux and all other kinds of digestive discomfort. Babies' Magic Tea is an herbal medicinal tea used for Babies to ease their digestive discomfort and help them sleep better and feel better

### DESCRIPTION

The present invention relates to a proprietary blend of herbal tea for babies. My invention was not created under any kind of government contract.

The proprietary blend contains two ingredients, organic cumin seeds and organic funnel seeds. In one embodiment, the proprietary blends contains 60% organic cumin seeds and 40% organic funnel seeds.

The herbal tea is caffeine free, all natural and organic.

The combination of the two herbs when used as a tea for babies, with the right amount of cumin seeds and the right amount of funnel seeds, is a very effective natural remedy for baby colic, gas, constipation and acid reflux.

Babies' Magic Tea is an all natural solution for babies with typical digestive problems.

From colic, gas, constipation, acid reflux, indigestion and other tummy troubles—it naturally soothes and comforts the child's belly issues. They'll sleep better, eat better and behave better when they're fully rested.

The preparation process: (1) Selection of raw materials; (2) Washed thoroughly; (3) air-dry sterilization; (4) ingredients; (5) mixed; (6) Tea bagged in 1.5 gram tea bags; (7) Every 20 tea bags are packed separately in BPA free, food grade packaging bags; (8) Every bag of 20 tea bags is packed in a tin can; (9) Every tin can is sealed in full with a plastic shrink wrap how to use Babies Magic Tea: boil 8 ounces of water, add the tea bag to boiling water, boil for 2 to 3 minutes, remove the tea bag and serve at room temperature

Serving suggestions:

1—Serve herbal baby tea as is

2—Mix herbal baby tea with formula

3—Mix herbal baby tea with breast milk

The tea is packages using the BPA free packaging bags and tea tin.

1. Tea comprising:
a predetermined amount of organic funnel seeds;
a predetermined amount of organic cumin seeds;
the tea comprising no other ingredients.

2. The tea of claim 1, wherein the predetermined amount of organic funnel seeds is 40% and the predetermined amount of organic cumin seeds is 60%.

3. The tea of claim 2, wherein the tea is intended for babies to reduce colic, gas, constipation, and acid reflux.

4. The tea of claim 3, wherein the tea may be mixed with baby formula and/or breast milk.

5. The tea of claim 4, wherein the tea functions as a sleep remedy.

6. A method of preparing tea comprising steps:
(a) selecting raw materials, wherein the raw materials include organic cumin seeds and organic funnel seeds;
(b) washing the raw materials thoroughly;
(c) sterilization of the raw materials via air-drying;
(d) mixing a predetermined amount of organic funnel seeds and a predetermined amount of organic cumin seeds into a tea bags, wherein the tea bag is 1.5 g;
(e) boiling 8 ounces of water, wherein the tea bag is added to the boiling water for 2 to 3 minutes; and
(f) serving the tea to a baby at room temperature wherein the tea is intended for babies to educe colic, gas, constipation, and acid reflux.

7. The method of claim 6, in step (f), wherein the tea may be mixed with baby formula and/or breast milk.
